(19) United States
(12) Patent Application Publication
(10) Pub. No.: US 2016/0236155 A1
(43) Pub. Date: Aug. 18, 2016

(54) MICROBIAL FILTER DEVICE AND METHOD FOR PROVIDING SUCH DEVICE

(71) Applicant: METALMEMBRANES.COM B.V., Leeuwarden (NL)

(72) Inventors: Sybrandus Jacob Metz, Leeuwarden (NL); Hans Hendrik Wolters, Leeuwarden (NL)

(73) Assignee: METALMEMBRANES.COM B.V., Leeuwarden (NL)

(21) Appl. No.: 15/024,580

(22) PCT Filed: Sep. 26, 2014
(86) PCT No.: PCT/NL2014/050659

§ 371 Date: Mar. 24, 2016

(30) Foreign Application Priority Data
Sep. 27, 2013 (NL) 2011516

(51) Int. Cl. B01D 69/12; C12Q 1/04; B01D 67/00; B01D 71/02; B01D 61/18; B01D 65/02

(52) U.S. Cl. CPC B01D 69/12; B01D 61/18; B01D 65/02; B01D 67/0062; B01D 71/022; B01D 71/024; C12Q 1/04; B01D 2321/22; B01D 2325/04

(57) ABSTRACT

The invention relates to a microbial filter device and method for providing such device. The method for manufacturing the microbial filter device comprises the steps of:—providing a first metal layer;—providing a side of the first metal layer with a porous metal oxide layer; and—after providing the porous metal oxide layer providing a number of chamber defining structures in the first metal layer that are in contact with the porous metal oxide layer.

The present invention relates to a microbial filter device. Such filter device allows for the growth of micro-organisms in a natural environment, for example

Conventional microbial filters that are known from practice comprise a membrane layer that is provided with a pore size which is permeable for nutrients and impermeable for micro-organisms. In principle such membrane allows for the growth of micro-organisms in a natural environment. Membrane materials that are used are preferably flat such as inorganic membrane materials that are well suited for a right range of laboratory filtration applications, such as an anopore membrane. To enable growth of micro-organisms in the filter, structures have to be made on the surface of the membrane(s).

Such conventional microbial filters are described in US 2006/0252044 A1, for example.

These conventional microbial filters require an additional manufacturing operation in order to provide such structure on the membrane and to create compartments which confine micro-organisms. A further problem associated with providing such structures on a conventional membrane is that leakage may occur between the different compartments on the membrane. Such leakage may be due to poor adhesion of a polymer to a ceramic material, for example.

The object of the present invention is to provide a method for providing a microbial filter device that obviates or at least reduces the above problems.

This object is achieved with the method for manufacturing a microbial filter device according to the present invention, the method comprising the steps of:
providing a first metal layer;
providing a side of the first metal layer with a porous metal oxide layer; and
after providing the porous metal oxide layer providing a number of chamber defining structures in the first metal layer that are in contact with the porous metal oxide layer.

By starting with a first metal layer, for example a metal sheet with the required dimensions, a porous metal oxide layer can be provided on preferably one side of the first metal layer. Such metal oxide layer can be provided with a plasma oxidation process, for example. As the adhesion of such metal oxide layer to the first metal layer is excellent no substantial leakage will occur in practice. This improves the applicability of the microbial filter device according to the present invention as compared to conventional filter devices. Also, the sealing capabilities of the metal oxide layer to the first metal layer are excellent and enable use in an effective sampling device.

According to the invention the first metal layer can be provided with a number of chamber defining structure or structures that are in direct contact with the metal oxide layer. This number can be one or more. In this description will be referred to compartments in general. The chamber defining structures define cavities or compartments or pockets or other structures wherein micro-organisms may grow. According to the invention the structures are provided after arranging the porous metal oxide layer to the first metal layer. It has been found that such chamber defining structures can be etched into metals such that the structures can be used in a microbial filter device according to a presently preferred embodiment of the invention. This etching involves chemical etching or electro chemical etching, also referred to as electrochemical machining (ECM), including jet electrochemical machining (JET-ECM), thereby allowing for a precise, fast and reproducible local removal of material of the first metal layer. Surprisingly, in this etching process it was found that etching the first metal layer does not significantly influence the metal oxide layer. In fact, the metal oxide layer remains substantially intact whereas the metal is locally etched away. This enables an efficient and effective manufacturing of the microbial filter device according to the present invention.

In a presently preferred embodiment according to the present invention the metal oxide layer is provided on a side of the first metal layer involving a plasma oxidation process, more specifically a plasma electrolytic oxidation (PEO) process. By performing a plasma electrolytic oxidation process on the first metal layer locally the electric brake down potential of the oxide film on the metal layer is exceeded and discharges occur. Such discharges lead to a type of local plasma reactors, resulting in a growing oxide. This builds the desired structure for the membrane layer. The plasma electrolytic oxidation process creates very fine pores in the metal layer, thereby forming an oxide layer that contains small pores and can be used for separation processes, such as acting as a microbial filter. This method provides a membrane layer that can be made efficiently. Surprisingly, also the pore sizes of this membrane layer can be controlled more effectively and the desired characteristics for such membrane layer can be achieved more accurately. In addition, such membrane is more stable and robust as compared to the resulting membrane from conventional manufacturing methods as the mechanical strength of the metal oxide layer is significantly stronger. This increased strength has as one of its effects that cracking of the resulting membrane is less likely. A further advantage of the method according to the invention is that it enables the manufacturing of membrane material in a modular way. This enables providing complicated three-dimensional shapes of the microbial filter device.

Preferably, providing the chamber defining structures involves etching the first metal layer. This etching is performed after arranging the porous metal oxide layer on the first metal layer.

In a further preferred embodiment according to the present invention the method for providing a microbial filter device further comprises the step of providing a second metal layer on the other side of the metal oxide layer. Such second metal layer provides additional strength and stability to the microbial filter device. In addition to, or alternative to, the second metal layer a porous layer can be applied, such as filter paper.

In a further preferred embodiment according to the present invention the method further comprises the step of cleaning the surface of the porous metal oxide layer that acts as the membrane layer.

By cleaning the surface of the membrane layer effectively the effects of fouling can be significantly reduced. In a presently preferred embodiment this cleaning of the surface comprises electro-filtration. It has been found that the microbial filter device according to the present invention can be cleaned effectively by applying an electric potential on the device. For example, when the structure with the metal oxide layer is provided with a negative charge and there is provided an electrode with a positive charge that is preferably positioned opposite to the surface of the membrane layer, the fouling particles can be removed from the membrane surface by the resulting electrical field. An advantage with the microbial filter device and method according to the present invention is that the applied field strength in cleaning the membrane layer can be relatively small For example, the strength can be in the order of magnitude of a few Volts as the metal layer is attached to the metal oxide layer. This provides an efficient and effective cleaning of the surface of the membrane surface.

The cleaning step, preferably involving electro-filtration, can be advantageously applied to microbial filter devices. It will be understood that this cleaning step can also be applied to other filtration applications involving filter devices.

In a further preferred embodiment according to the present invention the method involves detecting micro-organisms by applying a fluorescent labeling process. As the metal oxide and metal layers are not auto-fluorescent the detection of micro-organisms via labeling with fluorescent dyes can be applied. This provides an effective detection of micro-organisms.

The present invention also relates to a microbial filter device, comprising:
a first metal layer; and
a porous metal oxide layer arranged on at least one side of the first metal layer, wherein the first metal layer comprises a number of chamber defining structures that are provided in the metal layer after arranging the porous metal oxide layer and that are in direct contact with the porous metal oxide layer.

Such device provides the same effects and advantages as those related to the method.

By forming the porous metal oxide layer directly on the first metal layer it will be understood that the different layers have excellent adhesion characteristics as compared to conventional filter devices that involve adhering two separate layers after their respective manufacturing. In the filter device according to the invention this achieves an excellent sealing and significantly reduces leakage problems.

With the microbial filter device according to the present invention providing the chamber defining structure can be done after providing the porous metal oxide layer, surprisingly without damaging this oxide layer. This enables an effective manufacturing process and a high quality microbial filter device.

As a further advantage of the microbial filter device according to the present invention, the filter device allows for detection of microorganisms via labeling with fluorescent dyes as the metal oxide layer and the metal layer are not auto-fluorescent. This provides a further advantage when detecting micro-organisms.

Preferably, the material for the first metal layer is chosen from the group of materials that is capable of forming a non-conductive oxide, like titanium, aluminum, magnesium, zirconium, zinc and niobium, or an alloy. Experiments have shown that the specific group of materials may provide a membrane with desired characteristics that can be manufactured in an efficient manner In a presently preferred embodiment the porous metal oxide layer comprises pores with a size in the range of 0-1 μm, preferably in a range of 0-500 nm, and are most preferably below 200 nm.

The use of membranes with pore sizes in the afore-mentioned ranges, most preferably at least below 500 nm, improves the effect of the microbial filter device as nutrients may flow through the membrane layer while micro-organisms are kept in the chamber defining structures on the metal oxide layer.

Preferably, the majority of the pores, preferably at least 75%, and more preferably at least 90%, is in the afore-mentioned range. It was shown that when manufacturing the microbial filter device according to the present invention controlled pore size can be achieved in an effective manner such that it is possible to improve the applicability of the filter in a separation process even further.

The thickness of the metal oxide layer is preferably between 0 and 150 μm and most preferably in the range of 50-70 μm. This thickness showed good results.

In a presently preferred embodiment according to the present invention, the chamber defining structure is configured to grow microorganisms.

By providing the chamber defining structure directly in the first metal layer, and in contact with at least a part of the surface of the porous metal oxide layer that acts as membrane, an effective manufacturing process can be performed resulting in the microbial filter device according to the present invention. In a presently preferred embodiment the chamber defining structure is etched into the first metal layer after a porous metal oxide layer has been provided on one side of this metal layer.

Preferably, the chamber defining structure has a width or diameter for an individual structure or chamber in a range of 0-1 cm, preferably 1 mm-5 mm, and most preferably 2-4 mm In another preferred embodiment the chamber has a diameter in the range of 0-1 cm, preferably 0-1 mm, and most preferably 20-100 micrometer.

It has been shown that dimensions in the afore-mentioned ranges enable growth of micro-organisms in the chambers while still enabling liquid flow through the membrane formed by the metal oxide layer. Furthermore, these dimensions for a chamber enable access for a pipette. The structures may have any kind of shape, such as a square, circular or rectangular shape. Optionally, the distances between individual structures can be varied. Also, the material between individual structures can be provided with additional messages, codes etc. This provides an effective microbial filter device. In an embodiment according to the invention the filter device has a length of about 76 mm and a width of about 26 mm corresponding to the dimensions of a slide of a microscope.

In a further preferred embodiment according to the present invention, the microbial filter device further comprises a second metal layer provided on at least a part of the surface of the porous metal oxide layer.

Providing a second metal layer provides additional stability and/or strength to the microbial filter device. Such layer protects the metal oxide layer. Furthermore, this second metal layer may involve a porous metal layer such that flow is capable of flowing through the second metal layer. In addition to, or alternative to, the second metal layer a porous layer can be applied, such as filter paper.

Further advantages, features and details of the invention are elucidated on the basis of preferred embodiments thereof, wherein reference is made to the accompanying drawings, wherein:

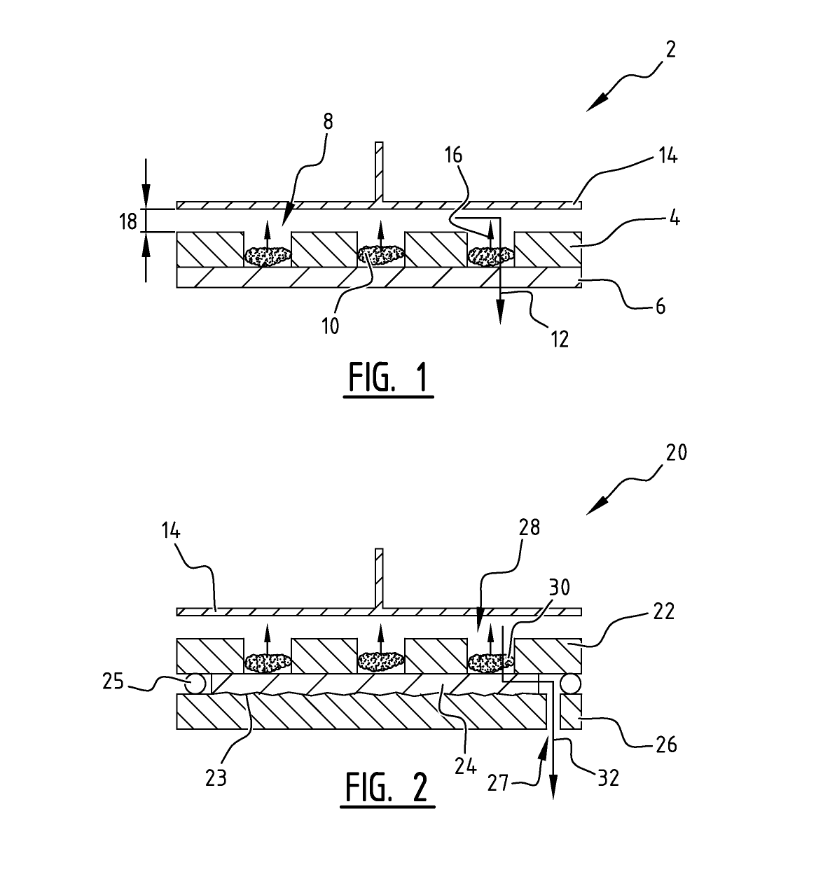

FIG. 1 shows a microbial filter device according to the invention;

FIG. 2 shows an alternative microbial filter device according to the invention;

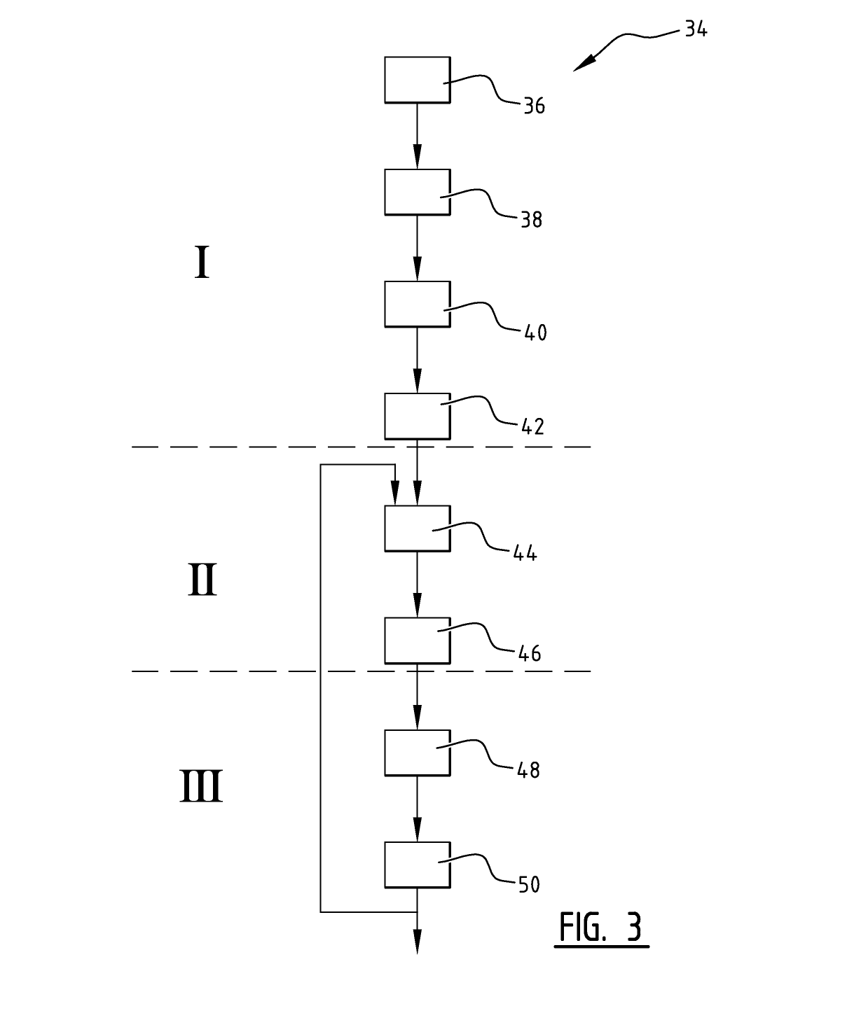

FIG. 3 illustrates the method steps according to the invention for manufacturing a microbial filter device according to the invention; and

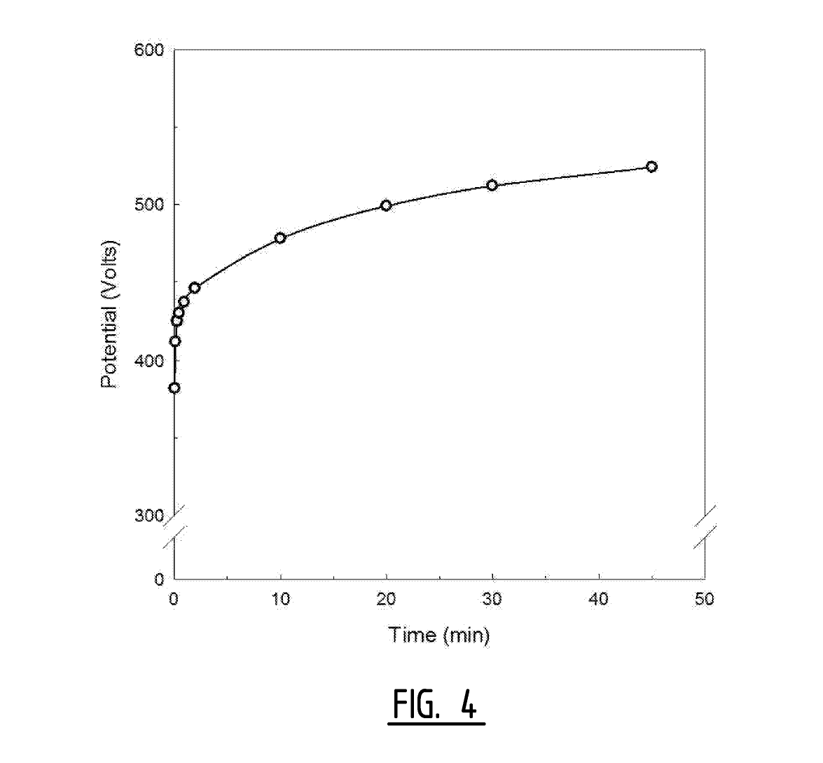

FIGS. 4-7 show experimental results with the device of FIGS. 1-3.

A membrane 2 (FIG. 1) comprises first metal layer 4. On one side of the metal layer 4 a metal oxide layer 6 acting as a membrane is formed with Plasma Electrolytic Oxidation (PEO). After forming oxide layer 6, metal layer 4 is provided with chambers 8 with ElectroChemical Machining (ECM), wherein micro-organisms 10 can be captured and may grow. In the illustrated embodiment liquid flow 12 flows through chambers 8 and through metal oxide layer 6.

In the illustrated embodiment metal layer 4 is provided from aluminum or titanium and metal oxide layer 6 comprises aluminum oxide or TiO₂ or Al₂O₃. It is understood that other materials are also possible in accordance with the present invention.

In an alternative embodiment filter device 20 (FIG. 2) comprises a first metal layer 22 and a metal oxide layer 24 that is formed thereon by PEO. Furthermore, device 20 comprises a second metal layer 26 on the other side of oxide layer 24 as an additional support layer with opening 27. Layer 26 is attached to first layer 22 with glue or welds 25. First metal layer 22 comprises chambers 28 that are provided using ECM wherein micro-organisms 30 are captured and may grow. It is noted that in the illustrated embodiment support layer 26 is not etched.

In this illustrated embodiment liquid flow 32 partly flows through membrane layer 24 and leaves filter device 20 through opening 27. It will be understood that other configurations are also possible. For example, second metal layer 26 can be provided with a porous structure such that flow 32 may pass directly through membrane layer 24 and second metal layer 26.

In the illustrated embodiment an additional electrode 14 is provided on the other side of metal layer 4, 26. Metal layer 4, 26 is provided with a negative charge and electrode 14 provided with a positive charge such that an electric field results. In this electric field, micro-organisms 10 start moving in direction 16 such that chambers 8 and membrane layer 6 can be cleaned effectively. Electrode 14 is provided at a distance 18 from the surface of metal layer 4, 26. When providing a charge to electrode 14 and metal layer 4 organisms 10 start to move in direction 16 away from membrane layer 6. Optionally, distance 18 is defined by the thickness of a glue line or adhesive layer.

In a manufacturing process 34 (FIG. 3) phase I starts with providing a first metal layer 4, 22 in step 36. Metal layer 4, 22 can be a metal sheet or metal foil, for example. In the illustrated embodiment a plasma oxidation process 38 is performed to achieve the metal oxide layer 6, 24 acting as membrane. Oxide layer 6, 24 has a roughness 23. Optionally a second metal layer 26 is provided in step 40. In step 42 electro-chemical etching is performed to provide the chamber defining structures 8, 28 on first metal layer 4, 22. This provides a microbial filter device 2, 20 according to the invention.

In measuring phase II microbial filter device 2, 20 is used and a flow is provided through membrane layer 6, 24 in step 44. In measuring step 46 the amount and/or type of micro-organisms is measured, for example using a fluorescent dye.

In cleaning phase III an additional electrode 14 is provided in first cleaning step 48. In second cleaning step 50 a charge is applied to the metal layer and the additional electrode 14. In this electro filtration process, micro-organisms or other fouling 10, 30 which remains on the membrane surface are removed from the surface of oxide layer 6, 24 such that the microbial filter device 2, 20 is cleaned.

Experiments with filter device 2, 20 show good results. Filter device 2, 20 can be manufactured effectively and performs advantageously without significant leakage problems. Cleaning with an electro-filtration step cleaned the membrane surface effectively.

As an example, manufacturing of a device that was used in the above experiments will be described.

An aluminum plate with a thickness of 0.5 mm was treated in a plasma oxidation reactor. This plate was mounted in the reactor where one side of the plate was placed opposite a cathode. The other side was sealed from the electrolyte. One side of the plate was treated with plasma electrolytic oxidation (PEO). The electrolyte contained amongst others potassium hydroxide (KOH) and sodium silicate (Na₂SiO3 5H₂O) dissolved in water. A potential was applied between the aluminum and the cathode. The current density was kept constant in a range of 300-3500 A/m². The potential increased rapidly from over 300 Volt in the first minute till higher values in the range of 400-700 Volt in the final minutes of treatment.

After the plasma oxidation treatment the metal plate was transferred to an etch cell. In this cell the metal was etched via electrochemical machining. The plate was mounted in this cell with the metal side facing the cathode. This cathode consists partly of a metal and a plastic. The metal shape of the cathode determines the shape and dimensions which will be etched in the metal plate. A pulsed electric field is applied between the cathode and the anode (metal plate with on the other side the metal oxide layer). A highly conductive electrolytic flow was provided between the anode and the cathode. The potential difference between the anode and the cathode was in the beginning 10-15 Volts and increased gradually during the etching. The potential increases sharply when the metal is etched away and reaches the metal oxide layer. Then the process was stopped. The current density was kept at a value of about 250 kA/m². This process results in a metal plate with on one side a metal oxide layer and a structure etched in the metal. Fluids can be filtrated through the open structure in the metal. The metal oxide layer can be supported during filtration by a metal plate and/or a (paper) filter that is optionally provided in between the metal oxide layer and the metal plate. Because the surface roughness of the metal oxide layer is high the permeate water can flow easily away to the sides and can be separated from the feed water. This filtration configuration also allows for high filtration pressures over 5 bars.

A solution of e-coli was filtered on this filter. A second solution was also filtered over this filter containing a fluorescent molecule such a propidium iodide. Under a fluorescent microscope the microorganisms could be clearly detected and counted via specialized software. In another configuration round pockets with a diameter of 2 mm were etched electrochemically as described before. In the pockets microorganism were inoculated. The membrane was placed with the metal oxide layer facing the growth medium which was optimal for the microorganisms. Transport of nutrients occurred through the metal oxide layer from the medium to the microorganisms and the microorganisms were kept in the pocket since the pore size of the metal oxide layer was smaller (<200 nm) than the microorganisms.

Small compartments or pockets in such configuration with a size smaller than 100 or even 50 micrometer can be electrochemically etched using jet electrochemical machining (JET-ECM), also referred to as electrolytic jet machining. An electrolytic current between the anodic work piece and the cathodic nozzle is supplied via an electrolyte jet which is ejected from a nozzle.

In another configuration microorganism were filtrated with the membrane which was produced as described above. After a period of filtration the bacteria and other components (particles and molecules) were retained on the membrane surface. This formed a cake-layer which reduced the transport through the membrane considerably. An electrical potential was applied between the membrane and a plate which was facing the feed-side of the membrane. The membrane was charged negatively (3 Volt) and the plate positively. Due to the electrical field the negatively charged cake layer was migrating away from the membrane surface toward the positively charged electrode. This caused an increase in flux 10 to 20 time higher as compared to when no electric field was applied. The advantage of the membrane described here is that the electric field can be relatively small (few Volts), thereby limiting electric consumption and electrode reaction.

Next some examples will be presented for the manufacturing and the use of the filter device according to the present invention. It will be understood that these examples illustrate aspects, details and features that may be applied in different combinations to other embodiments.

### EXAMPLE 1

### Ceramic Membrane Made Out of Alumina For Bacterial Analysis and Filtration Without Support Layer

An Aluminum plate of 0.5 mm thickness is used for this experiment.

Step 1: Plasma Electrolytic Oxidation (PEO). An electrolyte of 4 g/l Na₂SiO₃ 5H₂O and 2 g/l KOH is used. The Aluminum plate is mounted in a reactor where the electrolyte flows over the aluminum plate. This plate is used as an anode. The electrolyte is cooled through a heat exchanger.

A cathode is positioned at a short distance from the anode. The experiment is performed at constant current mode of 950 A/m². The potential difference between the anode and cathode increases in time. The increase in potential is shown in FIG. 4.

The plasma electrolytic oxidation is stopped after 45 minutes. The thickness of the porous layer ceramic layer on the metal plate is now 50 micron as measured by a thickness meter. In order to function as a filter the metal on the other side of the plate has to be removed (partially) in order to filtrate a fluid through the ceramic layer.

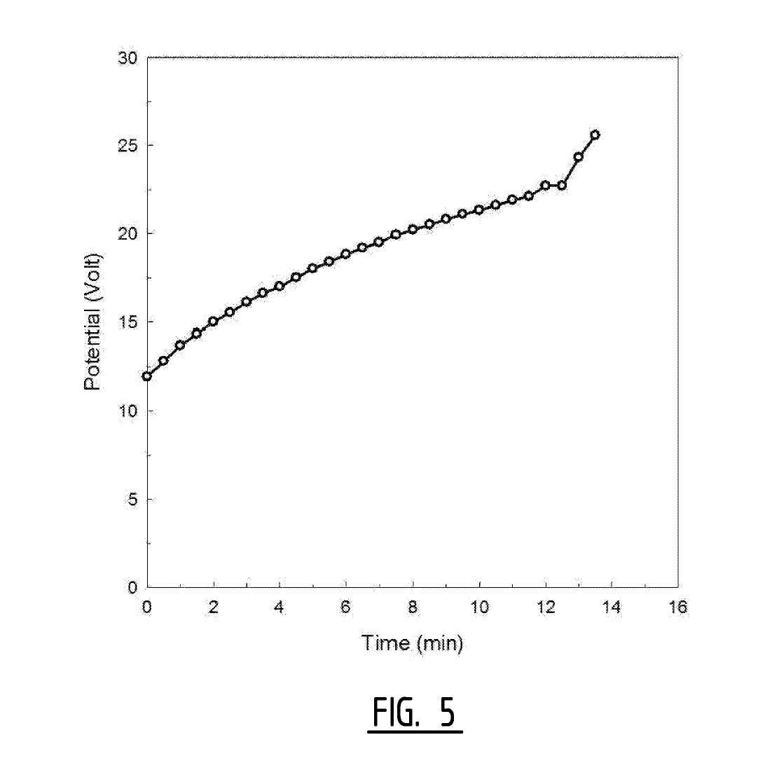

Step 2: Electrochemical Machining (ECM). In ECM the work piece (metal plate with oxide layer) is used as an anode. In the ECM process, a cathode (tool) is advanced into an anode (work piece). A highly conductive electrolyte of NaNO₃ (5 mole/l) flows between the anode and cathode. The material from the work piece is dissolved, as the tool forms the desired shape in the work piece. The electrolytic fluid carries away the metal hydroxide formed in the process. This process continues until the ceramic layer is reached. In this example is the distance between the anode an cathode fixed. The potential difference between anode and cathode increases in time due to the larger distance between anode and cathode. The increase is higher when the ceramic layer is reached. This potential increase between anode and cathode as function of time is shown in FIG. 5.

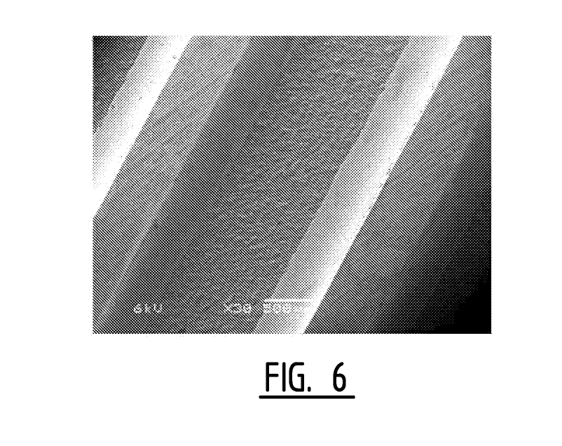

After ECM an Aluminum plate is obtained with flow channels with a ceramic layer (FIG. 6).

This filter is used to filter a solution containing bacteria. The bacteria are labelled with a fluorescent dyes and analyzed with a microscope. Bacteria can easily be identified since the membrane is not auto fluorescent. Since the bounding between the ceramic layer and the metal plate is excellent (ceramic layer is made out of the metal) no leakage can occur and attaching the plate to a filtration or sensor device is straightforward.

### EXAMPLE 2

### Membrane Filter With Support Plate

A membrane module is made through PEO and ECM from aluminum as described in Example 1. In a first step PEO achieves oxide layer 24 on first metal layer 22. However, before ECM is applied a (second) metal plate 26 is attached on the other side of the ceramic layer (the air side, where the biggest pores in the ceramic layer are). In this support plate 26 one or more holes 27 are drilled which can function as a permeate channel.

The roughness 23 of the ceramic layer 24 (on the side where it is connected to the other plate 26) is high enough to allow for flow of filtered water between the ceramic layer and attached metal plate 26. After performing ECM on first metal layer 22 providing the flow channels/cavities 28, the membrane is used as a filter with pressure of up to 3 bars.

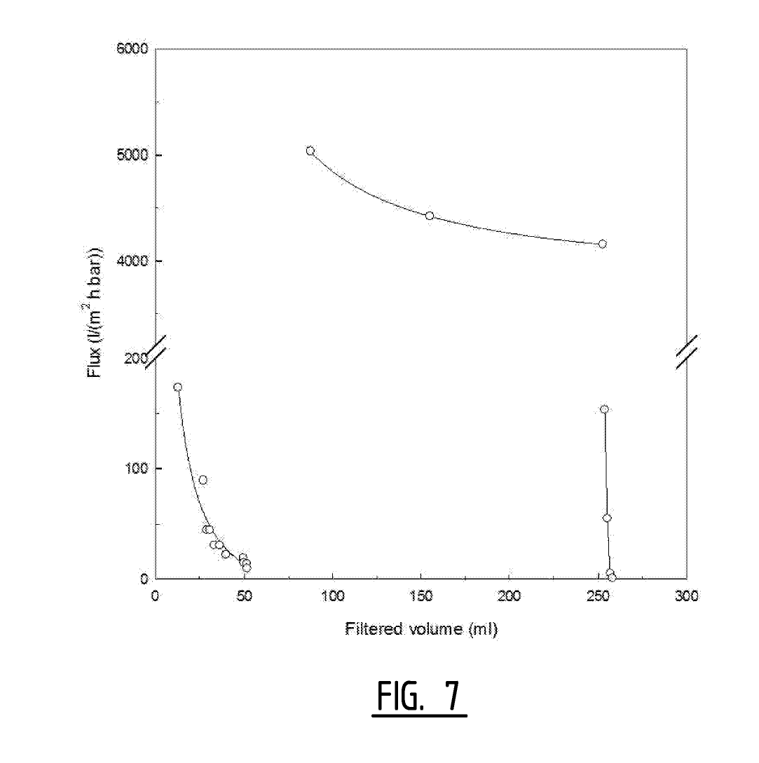

This membrane is used for filtration tests of which some results are shown in FIG. 7 showing fluxes of water for a fouled membrane as a dead-end filtration at 3 bar (left-below in FIG. 7), cleaned membrane (right-high in FIG. 7) and one with yeasts cells (right-below in FIG. 7).

The flux of the membrane at 3 bar declined due to fouling of the membrane in dead-end filtration. The membrane can withstand this high pressure due to the support plate which supports the ceramic layer. The membrane is cleaned with an NaOH solution 2 g/l for 30 minutes. The clean water flux of this membrane is high 4-5 m³/(m² h bar). After clean water filtration tests, a yeast cell solution 2 g/l was filtered. The flux dropped immediate due to fouling of the membrane surface.

### Example 3

### Electrofiltration

The porous ceramic layer 4, 24 is supported by a plate 26 (FIG. 2). This plate can be used as a cathode by giving it a negative charge. If an anode 14 is positioned above the first metal layer 4, 22 on the other side of membrane layer 6, 24 an electric field can be applied over the ceramic layer. This electric field can remove the fouling layer. This phenomenon is known in literature as electrofiltration.

The advantage of this module configuration is that no extra cathode has to be applied, since there is already a support plate which can be used as a cathode. A dimensions stable anode has to be used as an extra component in the cell. This anode can be positioned at a short distance from the ceramic layer, thereby lowering the energy consumption for electrofiltration. The thickness of the metal (which is connected to the porous ceramic layer) can be very thin <0.5 mm Therefore, anode 14 can be positioned close to the membrane surface creating a compact module configuration.

With a current density of 200 A/m² the fouling layer could be removed during filtration of a yeast cell solution. The flux with yeast cells (1 g/l) is 0.7 g/min With electric field it is 2.9 g/min comparable to the clean water flux. This shows that this cell configuration is suitable for electrofiltration.

The present invention is by no means limited to the above described preferred embodiments thereof. The rights sought are defined by the following claims, within the scope of which many modifications can be envisaged.

1. Method for manufacturing a microbial filter device, comprising the steps of:
providing a first metal layer;
providing a side of the first metal layer with a porous metal oxide layer; and
after providing the porous metal oxide layer providing a number of chamber defining structures in the first metal layer that are in contact with the porous metal oxide layer.

2. Method according to claim 1, wherein providing the metal oxide layer comprises a plasma oxidation process.

3. Method according to claim 1, wherein providing chamber defining structures in the first metal layer comprises etching the first metal layer.

4. Method according to claim 3, wherein the etching involves electrochemical machining.

5. Method according to claim 1, further comprising the step of providing a second metal layer on the other side of the metal oxide layer.

6. Method according to claim 1, further comprising the step of cleaning the surface of the porous metal oxide layer.

7. Method according to claim 6, wherein the cleaning of the surface comprises electro-filtration.

8. Method according to claim 7, wherein electro-filtration comprises providing a charge on the first metal layer.

9. Method according to claim 7, wherein electro-filtration comprises providing an additional electrode positioned substantially opposite to the surface of the porous metal oxide layer.

10. Method according to claim 1, further comprising detecting micro-organisms by applying a fluorescent labeling process.

11. Microbial filter device, comprising:
a first metal layer; and
a porous metal oxide layer arranged on at least one side of the first metal layer, wherein the first metal layer comprises a number of chamber defining structures that are provided in the metal layer after arranging the porous metal oxide layer and that are in direct contact with the porous metal oxide layer.

12. Microbial filter device according to claim 11, wherein the material for the first metal layer is chosen from the group of titanium, aluminum, magnesium, zirconium, zinc and niobium and/or an alloy.

13. Microbial filter device according to claim 11, wherein the porous metal oxide layer comprises pores with a size or thickness in the range of 0-1 μm, preferably in a range of 0-500 nm, and are most preferably below 200 nm.

14. Microbial filter device according to claim 13, wherein the majority of the pores, preferably at least 75%, and more preferably at least 90%, is in the specified range.

15. Microbial filter device according to claim 11, wherein the metal oxide layer has a thickness preferably between 0 and 150 μm and most preferably in the range of 50-70 μm.

16. Microbial filter device according to claim 11, wherein the chamber defining structure is configured to grow micro-organisms.

17. Microbial filter device according to claim 16, wherein the chamber defining structure has a width or diameter in the range of 0-1 cm, preferably 1 mm-5 mm, and most preferably 2-4 mm.

18. Microbial filter device according to claim 16, wherein the chamber defining structure has a width or diameter in the range of 0-1 cm, preferably 0-1 mm, and most preferably 20-100 micrometer.

19. Microbial filter device according to claim 11, further comprising a second metal layer provided on at least a part of the surface of the porous metal oxide layer.

20. Method for manufacturing a microbial filter device, comprising the steps of:
providing a first metal layer;
providing a side of the first metal layer with a porous metal oxide layer; and
after providing the porous metal oxide layer providing a number of chamber defining structure in the first metal layer that are in contact with the porous metal oxide layer,
wherein providing the metal oxide layer comprises a plasma oxidation process,
wherein providing chamber defining structures in the first metal layer comprises etching the first metal layer, and
wherein the etching involves electrochemical machining.
